(19) United States
(12) Patent Application Publication
(10) Pub. No.: US 2026/0087711 A1
(43) Pub. Date: Mar. 26, 2026

(54) METHODS, APPARATUS FOR SPECTRAL CT IMAGING AND CT SCANNING IMAGING SYSTEMS

(71) Applicant: WUHAN UNITED IMAGING LIFE SCIENCE INSTRUMENT CO., LTD, Wuhan, Hubei (CN)

(72) Inventor: Ming Ai, Wuhan, Hubei (CN)

(21) Appl. No.: 19/304,688

(22) Filed: Aug. 20, 2025

Related U.S. Application Data
(63) Continuation-in-part of application No. PCT/CN2024/077702, filed on Feb. 20, 2024.

(30) Foreign Application Priority Data
Feb. 20, 2023 (CN) 202310138044.X

(51) Int. Cl. G06T 11/00; A61B 6/00; A61B 6/03; G06T 5/50; G06T 7/00

(52) U.S. Cl. CPC G06T 12/30; A61B 6/032; A61B 6/5205; G06T 5/50; G06T 7/0014; G06T 12/10; G06T 2207/20221; G06T 2207/30096; G06T 2210/41; G06T 2210/52

(57) ABSTRACT

This disclosure relates to the field of X-ray based medical imaging technology, providing a method, device, and CT scanning imaging system for spectral CT imaging, which may improve the efficiency of reconstructed images. In this disclosure, after obtaining multiple data frames collected by the detector, each data frame is cached in energy segments and stored in memory; Read the data frames required for image reconstruction from memory or disk; Obtain reconstructed images using the read data frames.

### CROSS-REFERENCE TO RELATED APPLICATION

This application is a continuation-in-part of the international patent application PCT/CN2024/077702 filed on Feb. 20, 2024 in the United States. The international patent application claims priority to the Chinese patent application with application No. 202310138044. X, filed on Feb. 20, 2023 at the China Patent Office. The content of the above application is hereby fully incorporated as a reference.

### TECHNICAL FIELD

The present disclosure relates to the field of X-ray based medical imaging technology, and in particular, to methods, apparatus for spectral CT imaging and CT scanning imaging system.

### BACKGROUND

Over recent years, CT technology has advanced rapidly. By providing multi-dimensional and quantitative tissue-characterization information unavailable from conventional CT, spectral CT has emerged as a key future direction in the CT field. Spectral CT systems based on photon-counting detectors represent a new frontier in X-ray imaging technology. These systems offer several advantages, including reduced radiation dose and lower noise. In particular, photon-counting detectors introduce the concept of “energy bins,” enabling simultaneous acquisition of CT data at multiple energy thresholds for multi-energy imaging. While CT is now widely used in clinical diagnostics, spectral micro-CT systems that also employ photon-counting detectors are increasingly applied in pre-clinical research. Such systems demand high spatial resolution, long acquisition times, and generate large data volumes. Consequently, how to improve reconstruction efficiency and image quality remains a problem to be solved.

### SUMMARY

An aspect of the present disclosure may provide a method for spectral CT imaging, includes: obtaining a plurality of data frames collected by a detector; caching each of the data frames into memory in an energy-segmented manner, and thereafter storing the data frames in the memory to a storage device in an energy-segmented manner; reading, from the memory or the storage device, data frames required for image reconstruction; and generating a reconstructed image using the data frames that have been read.

In some embodiments, the method of reading, from the memory or the storage device, data frames required for image reconstruction includes: when the data frames required for image reconstruction reside in the memory, reading the data frames required for image reconstruction from the memory; and when the data frames required for image reconstruction do not reside in the memory, reading the data frames required for image reconstruction from the storage device.

In some embodiments, the method of generating a reconstructed image by using the data frames that have been read includes: storing the read data frames into shared memory; performing block-wise reading of the data frames in the shared memory to read at least one data frame at a time, thereby obtaining data frames for a plurality of projection angles; and reconstructing the data frames for the plurality of projection angles to obtain the reconstructed image.

In some embodiments, the method of the data frames for the plurality of projection angles include first-energy-segment data frames for the plurality of projection angles and second-energy-segment data frames for the plurality of projection angles, and wherein reconstructing the data frames for the plurality of projection angles to obtain the reconstructed image includes: reconstructing, by a first pipeline process, the first-energy-segment data frames for the plurality of projection angles to obtain a first-energy-segment reconstructed image; and reconstructing, by a second pipeline process, the second-energy-segment data frames for the plurality of projection angles to obtain a second-energy-segment reconstructed image, wherein the first pipeline process and the second pipeline process operate independently of each other.

In some embodiments, the method of reconstructing, by the first pipeline process, the first-energy-segment data frames for the plurality of projection angles to obtain the first-energy-segment reconstructed image includes: performing, by the first pipeline process, a correction process on the first-energy-segment data frames for the plurality of projection angles; and after the correction process, reconstructing, by the first pipeline process, the first-energy-segment data frames for the plurality of projection angles to obtain the first-energy-segment reconstructed image.

In some embodiments, the method for spectral CT imaging includes: before the step of performing, by the first pipeline process, the correction process on the first-energy-segment data frames for the plurality of projection angles, further includes: performing, by the first pipeline process, a fusion process on a plurality of first-energy-segment data frames of a same projection angle to obtain, for each projection angle, a fused first-energy-segment data frame.

In some embodiments, the method of the data frames for the plurality of projection angles include data frames corresponding to a plurality of energy segments for the plurality of projection angles, and wherein reconstructing the data frames for the plurality of projection angles to obtain the reconstructed image includes: performing, by a plurality of pipeline processes, image reconstruction on the data frames corresponding to the plurality of energy segments, thereby obtaining reconstructed images for the respective energy segments.

In some embodiments, the method further includes: displaying projection images corresponding to each angle or partial angle, sequentially, based on data frames read from memory or storage device at the same angle.

In some embodiments, the method of displaying projection images corresponding to each angle or partial angle, sequentially, includes: performing a correction process on first-projection-angle data frames read from the memory or the storage device to obtain and display a projection image corresponding to the first projection angle; and performing a correction process on second-projection-angle data frames read from the memory or the storage device to obtain and display a projection image corresponding to the second projection angle.

In some embodiments, the method of caching each of the data frames into memory in an energy-segmented manner and thereafter storing the data frames in the memory to a storage device in an energy-segmented manner includes: while writing a first batch of the plurality of data frames into the memory, writing a second batch of the plurality of data frames from the memory to the storage device.

In some embodiments, the plurality of data frames include full-energy-segment data frames and high-energy-segment data frames, the method further includes: obtaining low-energy-segment data frames based on a difference between the full-energy-segment data frames and the high-energy-segment data frames.

Another aspect of the present disclosure may provide an apparatus for spectral CT imaging, includes: an acquisition module configured to obtain a plurality of data frames collected by a detector; a storage module configured to cache each of the data frames into memory in an energy-segmented manner, and thereafter storing the data frames in the memory to a storage device in an energy-segmented manner; a reading module configured to read, from the memory or the storage device, data frames required for image reconstruction; and a reconstruction module configured to generate a reconstructed image by using the data frames that have been read.

Another aspect of the present disclosure may provide a CT scanning imaging system, includes: a detector configured to collect data frames after scanning; a memory cache unit configured to cache the data frames in an energy-segmented manner; a storage unit configured to store, in an energy-segmented manner, the data frames cached in the memory cache unit; and a processor configured to perform image reconstruction using data frames read from the memory cache unit or the storage unit.

Another aspect of the present disclosure may provide a computing device includes a processor and a memory storing computer program code, wherein the computer program code, when executed by the processor, causes the processor to perform the method described above.

Another aspect of the present disclosure may provide a non-transitory computer-readable storage medium storing computer program code, wherein the computer program code, when executed by a processor, causes the processor to perform the method described above.

Another aspect of the present disclosure may provide a computer program product storing computer program code, wherein the computer program code, when executed by a processor, causes the processor to perform the method described above.

The details of various embodiments of the present disclosure will be explained in the accompanying drawings and description below. Based on the description, drawings, and claims, those skilled in the art will easily understand other features, problems solved, and beneficial effects of the present disclosure.

### BRIEF DESCRIPTION OF THE DRAWINGS

FIG. 1 a flowchart illustrating a method for spectral CT imaging according to an embodiment;

FIG. 2 is a schematic diagram illustrating the workflow of data acquisition, caching and reading according to an embodiment;

FIG. 3 is a schematic diagram illustrating averaging processing of a plurality of data frames according to an embodiment.

FIG. 4 is a schematic diagram illustrating a multi-process pipeline framework according to an embodiment.

FIG. 5 is a schematic diagram illustrating an apparatus for spectral CT imaging according to an embodiment.

FIG. 6 is a schematic diagram of a CT scanning imaging system to an embodiment.

### DETAILED DESCRIPTION

In order that the objects, technical solutions and advantages of the present disclosure will become clearer, the present disclosure will be further described in detail below with reference to the accompanying drawings and embodiments. It should be understood that the specific embodiments described herein are merely for explaining the present disclosure and are not intended to limit the present disclosure.

In the present disclosure, the mention of “an embodiment” means that a particular feature, structure or characteristic described in connection with the embodiment is included in at least one embodiment of the present disclosure. The appearances of the phrase in various places in the specification are not necessarily all referring to the same embodiment, nor are they separate or alternative embodiments that are mutually exclusive with other embodiments. Person skilled in the art will explicitly and implicitly understand that the embodiments described in the present disclosure may be combined with other embodiments.

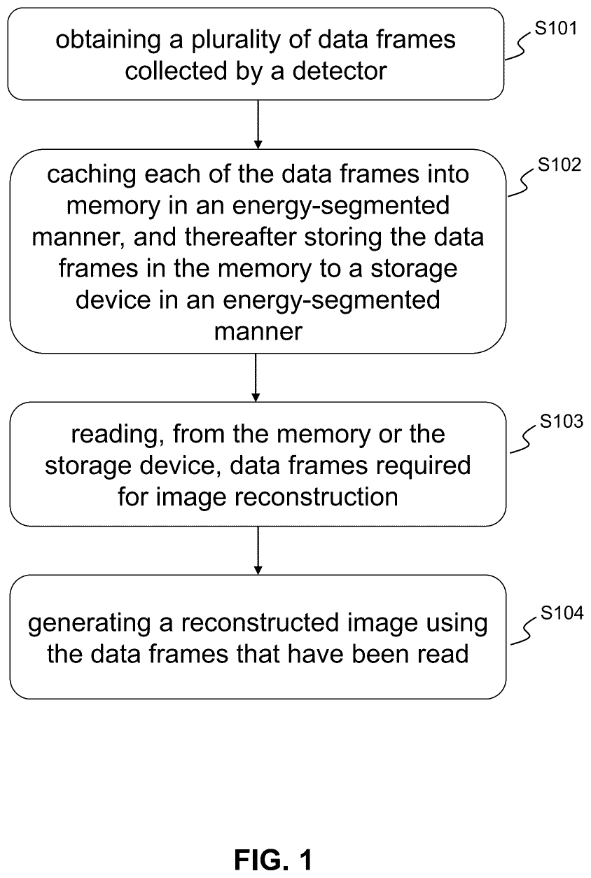

In an embodiment, as shown in FIG. 1, a method 100 for spectral CT imaging is provided, may include the following steps:

Step S101, obtaining a plurality of data frames collected by a detector. In conventional CT, the X-ray beam produced by the tube has a continuous energy distribution. Spectral CT imaging, utilizing the fact that different tissues exhibit different attenuation at different X-ray energies, provides more imaging information than conventional CT.

Spectral CT includes spectral Micro CT (micro-CT), which may be applied to pre-clinical research analysis, such as animal experiments. Spectral Micro CT has high requirements for spatial resolution, in some cases up to the micron level, and often adopts a step-and-shoot scanning mode to reconstruct images. In the step-and-shoot scanning mode involves rotating a specific angle to collect data once, completing the data collection at one angle and obtaining one or multiple data frames. In the step-and-shoot mode, data for one full rotation or multiple rotations may be acquired, or data for a non-integer rotation may also be acquired. If the rotation step angle is 1° and it takes 5 seconds to collect data once, and the data collected once is 360° for image reconstruction, it takes about 30 minutes to complete the collection.

The detector of energy spectrum CT may be a photon counting detector, which introduces the concept of “energy box” and may provide CT data with multiple energy thresholds simultaneously for multi energy imaging. Spectral CT may obtain data frames for different energy ranges by collecting data at different energy levels.

Step S102, caching each of the data frames into memory in an energy-segmented manner, and thereafter storing the data frames in the memory to a storage device in an energy-segmented manner.

A user may set a lowest energy threshold (generally higher than the background noise level of the detector), and the detector acquires photons whose energy is greater than or equal to the lowest energy threshold. The peak voltage of the X-ray source determines the maximum photon energy of the X-rays, which is an X-ray kVp (kilovolt peak). After the user sets the lowest energy threshold, the detector acquires photons whose energy is greater than or equal to the lowest energy threshold and less than or equal to the X-ray kVp, thereby obtaining full-spectrum data frames.

In some embodiments, for detectors having only two energy thresholds, a user may also set an intermediate energy threshold greater than the lowest energy threshold and less than the X-ray kVp. For example, if the user sets the lowest energy threshold to 20 keV and the X-ray kVp to 140 keV, the intermediate energy threshold may be set to 40 keV. After the intermediate energy threshold is set, the detector acquires photons whose energy is greater than or equal to the intermediate energy threshold and less than or equal to the X-ray kVp, thereby obtaining high-energy-segment data frames. The high-energy-segment data frames have an energy range of 40 keV-140 keV, while full-spectrum data frames have an energy range of 20 keV-140 keV. After obtaining the full-spectrum data frames and the high-energy-segment data frames, the low-energy-segment data frames may be obtained by subtracting the high-energy-segment data frames from the full-spectrum data frames.

It can be understood that the intermediate energy threshold set by the user may be one or more, and accordingly, multiple energy segment data frames may be obtained through collection or calculation.

After obtaining the plurality of data frames, the data frames are cached in the memory by energy segment. In the memory, data frames corresponding to the same energy segment are cached together. When data are acquired rapidly and a large volume of data is accumulated within a short time, the memory may be used to cyclically process the data in batches. When the cache size of a batch of data in the memory reaches a preset configuration size, the batch of data is sequentially stored into the storage device by energy segment. That is, while one batch of data is being written into the memory cache, the previous batch of data is being written into the storage device, and this process is repeated until data acquisition is complete.

A storage device may be a non-volatile storage device, for example, a disk. A disk is one of the most important storage devices in a computer. Most software required for normal operation of a computer is stored on a disk. Disk storage capacity is large, unlike memory. A disk is a storage device used in computers that stores and retrieves digital data using rapidly rotating disks (platters) with magnetic surfaces.
wherein, RAM (random access memory) is one of the important components in computers, serving as a bridge for communication with the CPU (central processing unit). The running of computer programs takes place in memory, so the performance of memory has a significant impact on the computer. Memory, also known as internal storage, is used to temporarily store computational data in the CPU and data exchanged with external storage such as hard drives. When the computer is running, the CPU transfers the data to be calculated to the memory for calculation. After the calculation is completed, the CPU transfers the result. The operation of the memory also determines the stable operation of the computer.

The storage device may be a non-volatile storage device, such as a disk. Disk is one of the most important storage devices in computers. Most of the software required for the normal operation of a computer is stored on disk. Disk storage has a larger capacity, which is different from memory. A disk is a storage device on a computer that is based on a hard rotating disk. It stores and retrieves digital data on a flat magnetic surface.

Step 103, reading, from the memory or the storage device, data frames required for image reconstruction.

After caching multiple data frames in memory according to energy segments and storing them in storage device, read data frames from memory or storage device based on whether the data frames required for image reconstruction are in memory. Taking the example of capturing a 360° data circle for image reconstruction at the same energy range with a rotation step angle of 1°, read the data frames corresponding to the same energy range at {1°, 2°, 3°, . . . , 360°} angles.

According to some embodiments of the present disclosure, during the scanning process, data frames are received from the detector and cached in memory. Write data from memory to storage device for long-term storage by blocks, while reading data from memory for online image reconstruction by blocks. Reading the data frame from storage device if the data frame is not in memory.

If the data frames required for image reconstruction are neither in memory nor in storage device, the corresponding pipeline process may automatically end due to data read timeout. In addition, if the data could not be read after reaching the waiting time, the Users may receive reminders through the interface that image reconstruction has failed. The user may click the reconstruction button again according to the timing to start the corresponding pipeline process in the backend.

Step 104, generating a reconstructed image using the data frames that have been read.

After reading the data frames, a preset reconstruction algorithm may be used to reconstruct the read data frames and obtain a reconstructed image.

According to some embodiments of the present disclosure, during the scanning process, data frames are received from the detector and cached in memory. The data frames are block-written from the memory into the storage device for long-term storage, while at the same time the data frames are block-read from the memory for on-line reconstruction. If the data frames are not in memory, they are read from the storage device. The online reconstruction process corresponds one-to-one with the scanning process, that is, each scan is followed immediately by an on-line reconstruction.

After scanning is completed, users may select different energy segment data frames or multiple energy segment data frames for multiple offline reconstructions as needed, and offline reconstructions do not rely on scanning. Offline reconstruction requires reading the data frames stored in the storage device into the memory first, and the subsequent data processing flow is the same as the online reconstruction flow, with corresponding correction and reconstruction processing. In addition, there is no projection image during the offline reconstruction process, and the projection image is displayed in real-time during the scanning process.

In the above methods, the decoupling design of data acquisition, storage, and reading is modularized to make each module function as single as possible, easy to expand and maintain; Firstly, in the case where the detector may generate multiple energy segment data frames in one acquisition, the data frames are parsed according to energy types and stored in different energy segments after recombination, which facilitates later viewing and application, and improves the efficiency of reconstructing the image; Secondly, in the case of a large amount of data during the same time period, a batch of data may be cached in memory first, and then stored in the storage device, greatly improving the speed of data writing to the storage device and enhancing the efficiency of image reconstruction; Next, read the data frames required for image reconstruction from the memory or the storage device, and output the reconstructed image.

In an embodiment, the step of reading, from the memory or the storage device, data frames required for image reconstruction, includes: when the data frames required for image reconstruction reside in the memory, reading the data frames required for image reconstruction from the memory; when the data frames required for image reconstruction do not reside in the memory, reading the data frames required for image reconstruction from the storage device.

From a hardware perspective, shared memory may be a part of the aforementioned memory, both of which are storage devices of the same nature and can be understood as physical memory storage devices. However, their functions are different. Shared memory allows two unrelated processes to access the same logical memory and is a shared memory space for cross process processing; Shared memory is a very effective way for two running processes to share and transfer data; The shared memory between different processes is usually the same physical memory segment; Processes may connect the same physical memory segment to their own address space, and all processes may access addresses in shared memory. If a process writes data to shared memory, the changes made may immediately affect any other process that may access the same segment of shared memory.

In some embodiments, if the data required for reconstruction are in the memory, they are preferably read from the memory into shared memory. If the data are not in the memory, they are read from the storage device into shared memory, to improve data reading speed.

In an embodiment, the step of generating a reconstructed image by using the data frames that have been read, includes: storing the read data frames into shared memory; performing block-wise reading of the data frames in the shared memory to read at least one data frame at a time, thereby obtaining data frames for a plurality of projection angles; and reconstructing the data frames for the plurality of projection angles to obtain the reconstructed image.

The above angle is a specific angle rotated to in step scanning mode. When reading data frames, it is possible to set at least one data frame to be read at a time, such as M data frames, where M is a natural number greater than or equal to 1. This reading method may be called block reading or batch reading. The blocks of data frames may be flexibly configured as needed, for example, all or part of the data frames corresponding to the same angle may be grouped as one block, or data frames corresponding to multiple angles may be grouped as one block. For example, some or all data frames corresponding to the same energy segment may be grouped as a block.

As mentioned above, during reconstruction, data may be collected based on the rotation angle set by the user. Generally, data may be collected for one rotation, or for scenes with less than or more than one rotation. The angle or number of rotations and step angle that need to be collected may be set by the user. After storing the read data frames in the shared memory, the data frames stored in the shared memory may be read in blocks to obtain data frames at angles such as {1°, 2°, 3°, . . . , 360°}. These angle data frames are then reconstructed to obtain reconstructed images.

In an embodiment, the step of reconstructing the data frames for the plurality of projection angles to obtain the reconstructed image, includes: reconstructing, by a first pipeline process, the first-energy-segment data frames for the plurality of projection angles to obtain a first-energy-segment reconstructed image; and reconstructing, by a second pipeline process, the second-energy-segment data frames for the plurality of projection angles to obtain a second-energy-segment reconstructed image, wherein the first pipeline process and the second pipeline process operate independently of each other.

In some embodiments, data processing in a pipeline process is performed by means of multi-thread parallel processing and pipeline communication, that is, data are divided into a plurality of threads from an input node through various intermediate computation processing nodes to an output node, and data between threads are communicated via pipeline sharing; the entire data processing process may be regarded as a pipeline.

The first energy segment may be any one of a high-energy segment, a full-spectrum segment, and a low-energy segment, and the second energy segment may be any one of a high-energy segment, a full-spectrum segment, and a low-energy segment, provided that the first energy segment and the second energy segment are different.

Taking the first energy segment as the full-spectrum segment and the second energy segment as the high-energy segment as an example.

For full-spectrum data frames at the angular positions {1°, 2°, 3°, . . . , 360°}, a pipeline process may be initiated to perform reconstruction processing on these data frames to obtain a full-spectrum reconstructed image. For high-energy-segment data frames at the angular positions {1°, 2°, 3°, . . . , 360°}, if hardware resources permit, another pipeline process may be initiated to perform reconstruction processing on these data frames to obtain a high-energy-segment reconstructed image. The two initiated pipeline processes are independent of each other.

In the present embodiment, creating a plurality of mutually independent pipeline processes not only enables parallel reconstruction processing of data frames of different energy segments, but also avoids mutual interference, ensuring high efficiency and timeliness of multi-energy-segment data processing.

In an embodiment, the step of reconstructing, by the first pipeline process, the first-energy-segment data frames for the plurality of projection angles to obtain the first-energy-segment reconstructed image, includes: performing, by the first pipeline process, a correction process on the first-energy-segment data frames for the plurality of projection angles; and after the correction process, reconstructing, by the first pipeline process, the first-energy-segment data frames for the plurality of projection angles to obtain the first-energy-segment reconstructed image.

In some embodiments, the correction processing includes at least one of air correction, geometric correction, bad-channel correction of the detector, focal-spot-position correction of the X-ray tube, and the like.

In this embodiment, the created pipeline process is not only used for reconstruction processing, but also for correction processing. For example, for the first energy segment data frames at {1°, 2°, 3°, . . . , 360°} angles, a pipeline process may be created to correct and reconstruct these data frames, obtaining the reconstructed image corresponding to the first energy segment.

In an embodiment, before the step of performing, by the first pipeline process, the correction process on the first-energy-segment data frames for the plurality of projection angles, the method further includes: performing, by the first pipeline process, a fusion process on a plurality of first-energy-segment data frames of a same projection angle to obtain, for each projection angle, a fused first-energy-segment data frame.

In this embodiment, when creating a pipeline process, the pipeline process may also be used for the above fusion processing. For example, after collecting multiple first energy segment data frames at angle a, the created pipeline process is used to fuse the multiple first energy segment data frames at angle a to obtain the first energy segment data frame at angle a.

It can be understood that in some embodiments, the present disclosure may also use multiple pipeline processes to perform image reconstruction on data frames corresponding to multiple energy segments, as described earlier, to obtain reconstructed images corresponding to each energy segment.

Further, a plurality of threads included in the pipeline process corresponding to the first energy segment may be used to perform fusion processing on first-energy-segment data frames at different angular positions in parallel, where one thread may also correspond to processing data frames at one angular position or at a plurality of angular positions. Through one thread, fusion processing is performed on first-energy-segment data frames at a specific angular position, so that fusion processing for different angular positions is performed in parallel. For example, after collecting multiple first energy segment data frames at angles a and b, the two threads included in the pipeline process corresponding to the first energy segment, such as the first thread and the second thread, are used to fuse the multiple first energy segment data frames at angles a and b, respectively, to obtain the first energy segment data frames at angles a and b. This enables parallel fusion processing for angle a and angle b. After collecting multiple first energy segment data frames at angles c and d, the first and second threads may also continue to fuse and process the multiple first energy segment data frames at angles c and d, respectively.

After obtaining the first energy segment data frames at {1°, 2°, 3°, . . . , 360°} angles, the pipeline process is used to perform correction and reconstruction on the first energy segment data frames at {1°, 2°, 3°, . . . , 360°} angles, obtaining the reconstructed image corresponding to the first energy segment.

In one embodiment, multiple angles of second energy segment data frames are reconstructed through a second pipeline process to obtain reconstructed images corresponding to the second energy segments, including: correcting multiple angles of second energy segment data frames through a second pipeline process. After correction processing, the second energy segment data frames from multiple angles are reconstructed through the second pipeline process to obtain the reconstructed images corresponding to the second energy segment.

In this embodiment, the created pipeline process is not only used for reconstruction processing, but also for correction processing. For example, for the second energy segment data frames at {1°, 2°, 3°, . . . , 360°} angles, a pipeline process may be created to correct and reconstruct these data frames, obtaining the reconstructed image corresponding to the second energy segment.

In one embodiment, before performing correction processing on the second energy segment data frames from multiple angles through the second pipeline process, the method provided by the present application further includes: obtaining the second energy segment data frames for each angle based on fusion processing of multiple second energy segment data frames from the same angle through the second pipeline process.

In the case of creating a pipeline process, the pipeline process may also be used for the above fusion processing. For example, after collecting multiple second energy segment data frames at angle a, the created pipeline process may be used to fuse the multiple second energy segment data frames at angle a to obtain the second energy segment data frame at angle a.

Furthermore, multiple threads contained in the pipeline process corresponding to the second energy segment may be utilized to fuse data frames from different angles of the second energy segment. One thread may also process data frames from one or multiple angles, and through one thread, fuse data frames from specific angles of the second energy segment to ensure parallel fusion processing for different angles. For example, after collecting multiple second energy segment data frames at angles a and b, the pipeline process corresponding to the second energy segment includes two threads, such as the third thread and the fourth thread, to fuse the multiple second energy segment data frames at angles a and b, respectively, to obtain the second energy segment data frames at angles a and b. This enables parallel fusion processing for angle a and angle b. After collecting multiple second energy segment data frames at angles c and d, the third and fourth threads may also be used to fuse and process the multiple second energy segment data frames at angles c and d, respectively.

After obtaining the second energy segment data frames at {1°, 2°, 3°, . . . , 360°} angles, the pipeline process is used to perform correction and reconstruction on the second energy segment data frames at {1°, 2°, 3°, . . . , 360°} angles to obtain the reconstructed image corresponding to the second energy segment.

When host hardware resources permit, a plurality of independent pipeline processes may be adopted, with each pipeline process corresponding to an energy segment, so that the reconstruction processing of data frames in different energy segments may be parallel and do not interfere with each other, ensuring efficient and timely processing of multi energy segment data frames.

In some embodiments, a multi-frame acquisition mode may be adopted, that is, when the object is rotated to a specific angular position, a plurality of data frames are acquired at the specific angular position. Through the multi-frame acquisition mode, it is possible to ensure data correction and image reconstruction quality even if the photon information collected in each data frame is relatively small, even with a small dose of energy spectrum Micro CT tube.

After collecting multiple data frames at a specific angle, multiple first energy segment data frames at the specific angle may be fused to obtain the first energy segment data frame at the specific angle, or multiple second energy segment data frames at the specific angle may be fused to obtain the second energy segment data frame at the specific angle.

In addition, it should be noted that in multi frame acquisition mode, that is, when the detector triggers the acquisition of multiple data frames from the same angle at once, these data frames may be quickly processed into a normal data frame before proceeding with the subsequent correction and reconstruction process. This may not affect the subsequent process. In some embodiments, data processing nodes for multi frame processing may be designed. Multi frame processing is the fusion processing of multiple data frames from the same angle. If only a single data frame from a certain angle is collected, multi frame processing may not be performed. Fusion processing methods include accumulating multiple data frames from the same angle and taking the average, or filtering a portion of each data frame according to certain rules, weighting and concatenating the filtered data, etc. By processing multiple frames, it is possible to ensure efficient data processing and improve the information content of photon data, thereby enhancing image quality.

In one embodiment, the method further includes: displaying projection images corresponding to each angle or partial angle, sequentially, based on data frames read from memory or storage device at the same angle. A projection image is an image of the original data that has been corrected but not reconstructed. Reconstructing an image generally requires scanning the set angle and accumulating data through a reconstruction algorithm. As the reconstructed image needs to wait for the scanning set angle to be completed, the process of generating the image is relatively time-consuming. For example, in the step scan mode, collecting 360° data with a rotation step angle of 1° takes 5 seconds to collect data at each step angle, and completing the set angle scan takes about 30 minutes. Therefore, obtaining the reconstructed image takes at least 30 minutes, and the software user interface may not display the reconstructed image during this process. By outputting the projection images corresponding to each angle, the projection images of the scan ned objects at the collected angles may be directly presented on the user interface, with high real-time performance and improved user experience.

Correction is the processing of data frames from a single angle, while reconstruction is the processing of data frames from multiple angles. After saving the correction processing results of each angle's data frame, notify the fore-end interface to load the projection image of this angle for display, in order to achieve the effect of fast image output. Similarly, after collecting data frames from one angle, notifying the fore-end interface to dynamically update and display the projection image corresponding to that angle may greatly improve the user interaction experience and avoid users waiting for multiple angle data frames to be collected and reconstructed before seeing the collected image effect. At the same time, the data frames of this process may be accumulated to the reconstruction processing node. After processing the data frames from multiple angles, the reconstructed image may be obtained and saved, and may be directly updated to the front-end interface for display. Alternatively, users may view and process them through image viewing software.

In one embodiment, displaying projection images corresponding to each angle or partial angle, sequentially, based on data frames read from memory or storage device at the same angle, including: performing a correction process on first-projection-angle data frames read from the memory or the storage device to obtain and display a projection image corresponding to the first projection angle; and performing a correction process on second-projection-angle data frames read from the memory or the storage device to obtain and display a projection image corresponding to the second projection angle.

The data frame of the first angle is collected first than the data frame of the second angle. This embodiment may display the projection images of each angle in the order of collection. For example, collecting 360° data with a rotation step angle of 1°, displaying projection images at 1°, 2°, 3°, . . . , 360° angles in sequence. Of course, in some embodiments, it is also possible not to display the projection images for each angle. Partial angle projection images may be displayed sequentially through internal algorithms or custom settings. For example, displaying projection images at angles of 1°, 5°, and 10° in sequence.

When rotating to the first angle, collect the data frame of the first angle, and rotate to the second angle to collect the data frame of the second angle. Wherein, after collecting the data frame of the first angle, the data frame of the first angle is corrected to obtain the projection image of the first angle and display it. After rotating to the second angle and collecting the data frame of the second angle, process the data frame of the second angle to obtain the projection image of the second angle and display it.

To better understand the above method, an embodiment is described in detail below. This embodiment provides a method for energy spectrum CT step-by-step multi frame scanning to obtain images, which adopts technologies such as multi frame processing, sequential storage, shared memory, block storage, and batch reading to improve data access efficiency. In addition, by utilizing the characteristic of energy spectrum scanning to collect multiple energy segment data in one scan, multi process pipeline and parallel computing technology are adopted to improve the quality and efficiency of reconstructed images.

When a plurality of data frames are acquired at an angular position, a certain algorithm is used to perform fusion processing on the plurality of data frames at the angular position, fusion processing performed on a plurality of data frames at the same angular position may be referred to as multi-frame processing. Fusion processing methods include accumulating multiple data frames from the same angle and taking the average, or filtering a portion of each data frame according to certain rules, weighting and concatenating the filtered data, etc.

When writing to the storage device, data are generally written in a certain order, and the order in which the storage device is written is referred to as storage order. When storing data frames, different data frames may be stored in different blocks, which may be called block storage. When reading data frames, it is possible to set M data frames to be read at once, which may be referred to as batch reading or block reading.

The step scan mode is generally used in Micro CT, which is characterized by rotating one angle to collect data once, and this scanning process takes a relatively long time. The conventional energy spectrum Micro CT based on photon counting detectors has many advantages such as multi energy imaging and precise quantitative analysis. However, due to the increase in data volume and uncertain factors affecting image quality parameters, it may affect the quality and efficiency of the image.

In the embodiments of the present disclosure, the data access of the energy spectrum CT step by step multi frame scanning method supports the reading and writing of multi energy segment data, and may efficiently perform parallel calculations and processing on different energy segment data. Some energy segment data may be obtained through calculations on other energy segment data, for example, low-energy segment data frames are equal to the full energy segment data frames minus the high-energy segment data frames.

In the embodiments of the present disclosure, data access also supports the acquisition mode of detector multi frame scanning. Collecting multi frame data may increase the amount of photon data information, which may improve image quality. In the embodiments of the present disclosure, the data multi frame processing method provided is based on the fusion processing of multiple data frames of the same energy segment at the same angle, parallel processing of multiple angles and energy segments, and obtaining data frames of each energy segment at each angle, without affecting the normal calibration and reconstruction process of subsequent data.

In addition, in the embodiments of the present disclosure, data access also supports a mode of writing and reading at the same time, which may quickly obtain a data frame of an angle in near real time, and then correct the data frame of this angle to obtain a projection image of this angle. The projection image of this angle is first presented on the interface. When continuing to rotate, as the angle changes, the interface also displays projection images of different angles, allowing users to feel the process of scanning and acquisition, improving the user experience. At the same time, during this process, the collected data frames are accumulated and multi energy segment image reconstruction is performed. After reconstruction is completed, the front-end interface may be notified to update and display the reconstructed image.

According to different embodiments of the present disclosure, the spectral CT imaging method has the following characteristics.

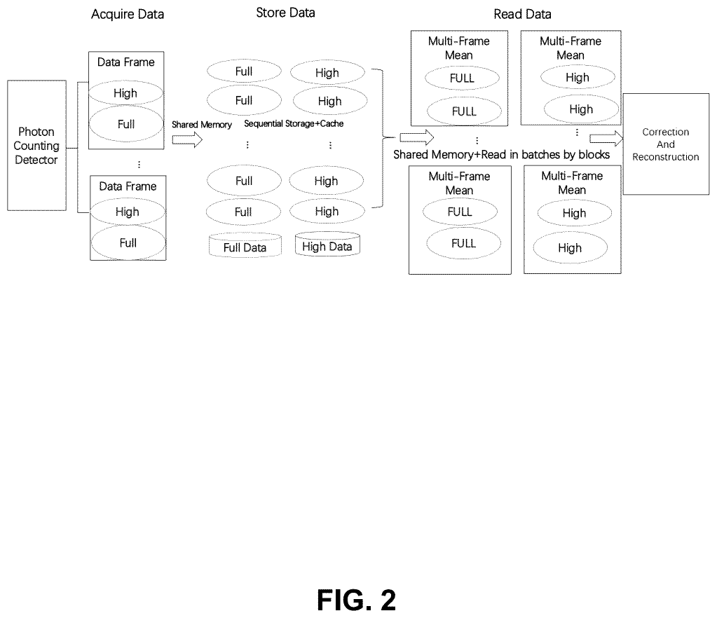

Adopting a modular decoupling design for data acquisition, storage, and reading, each module has a single function and is easy to expand and maintain. For example as shown FIG. 2, in the embodiments of the present disclosure, the photon counting detector may generate multiple energy segment data in one acquisition. Firstly, the data is parsed by energy type and then reassembled into different energy segment data frames for storage, including high-energy segment data frames and full energy segment data frames, for easy viewing and application later on. Secondly, using memory to first cache a batch of data and then sequentially store it in storage device ((e.g., hard disk) which greatly improves the speed of writing data into the storage device and ensures data integrity and no loss. Then, data are read from shared memory in blocks and in batches. If the data to be read are in the memory, they are preferably read from the memory into shared memory. And if not, they are read from the storage device into shared memory. The above methods in this embodiment may greatly improve the data reading speed and solve the problem of large data volume during the same time period.

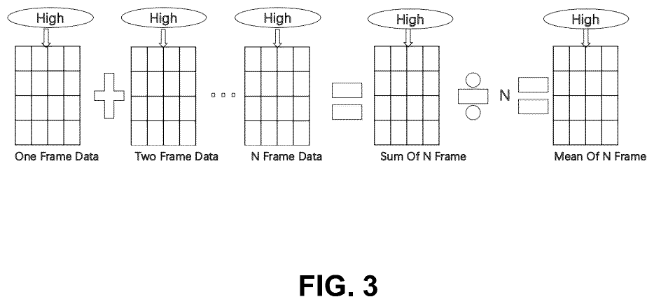

Design of a matrix parallel computation framework, capable of supporting maximized parallel processing of multi-frame data, and simultaneously supporting parallel computation processing of multi-energy-segment data, such as multi-frame data fusion processing. Refer to FIG. 3, image data are generally represented and processed in matrix form, and parallel computation processing may be performed on the data at each point of the matrix.

In the embodiments of the present disclosure, when in a scout scanning mode, based on the photon information at each point of the acquired data, radiation dose at different positions of different scanned objects may be intelligently analyzed and calculated, so as to determine whether the number of scanning frames set by the user is appropriate. If not, according to the number of scanning frames set by the user in subsequent tomographic scanning, automatic determination is made as to whether to adopt a single-frame acquisition mode or a multi-frame acquisition mode that may increase the number of photons. When it is automatically determined to adopt the single-frame acquisition mode, each time the object is rotated to a specific angular position, one data frame at the specific angular position is acquired. When it is automatically determined to adopt the multi-frame acquisition mode, each time the object is rotated to a specific angular position, N data frames at the specific angular position are acquired according to the number of frames N required by the user, where the number of frames N may be determined by an intelligent analysis algorithm.

In this way, the user does not need to be concerned about when to start the multi-frame acquisition mode, nor does the user need to attempt to set how many frames are appropriate, that is, throughout the scanning process, the user does not need to worry about the influence of the scanning frame parameter on image quality.

The above scout scanning refers to a scanning mode in which an X-ray tube and detector remain stationary, and the subject moves uniformly along with the examination table within the scanning bore, and the tube is simultaneously exposed, and a planar image is obtained.

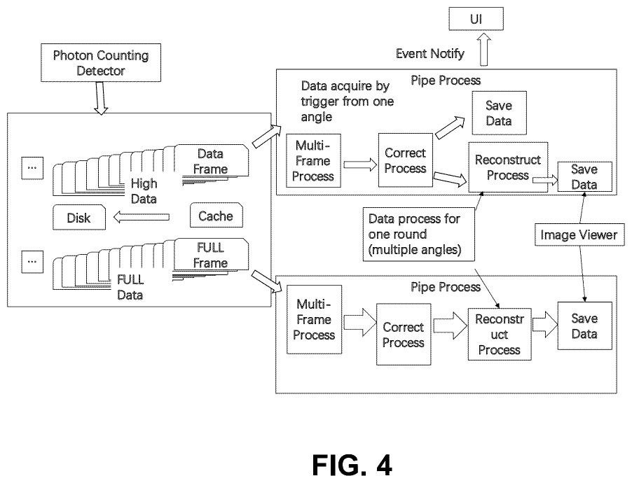

Design of a multi-process pipeline framework, capable of supporting real-time dynamic image output and simultaneously supporting maximized parallel processing of multi-energy-segment data. Refer to FIG. 4, while data are uniformly stored by energy segment type, as shown in FIG. 4 for high-energy-segment data frames, an independent pipeline process is initiated to perform correction processing on each angular position to form a projection image, and during generation of the projection image, appropriate window width and window level values are automatically calculated based on the data information acquired at each angular position, so that the projection image at each angular position may be adaptively displayed with window width and window level, greatly improving user experience.

Wherein, window width is the gray-level range displayed in a CT image, and window location is the center position of the window width. If the two values of window width and window location are not set properly, the display effect for normal tissues or lesions of different densities may be poor. Generally, in CT reconstructed images, common tissues have fixed ranges of window width and window location values. Sometimes the user needs to manually adjust window width and window location values to achieve optimal viewing. In the embodiments of the present disclosure, the system automatically calculates appropriate window width and window location values based on the gray-level distribution of the image, which may replace the manual adjustment process and improve user experience.

It should be understood that although the steps in the flowcharts described above are sequentially displayed according to the arrows, these steps are not necessarily performed in the order indicated by the arrows. Unless explicitly stated herein, there is no strict order limitation on the execution of these steps, and these steps may be performed in other orders. Moreover, at least some of the steps in the flowcharts described above may include a plurality of steps or stages, which are not necessarily performed at the same time, but may be performed at different times; the execution order of these steps or stages is not necessarily sequential, and at least part of these steps or stages may be performed alternately or in parallel with steps or stages in other steps.

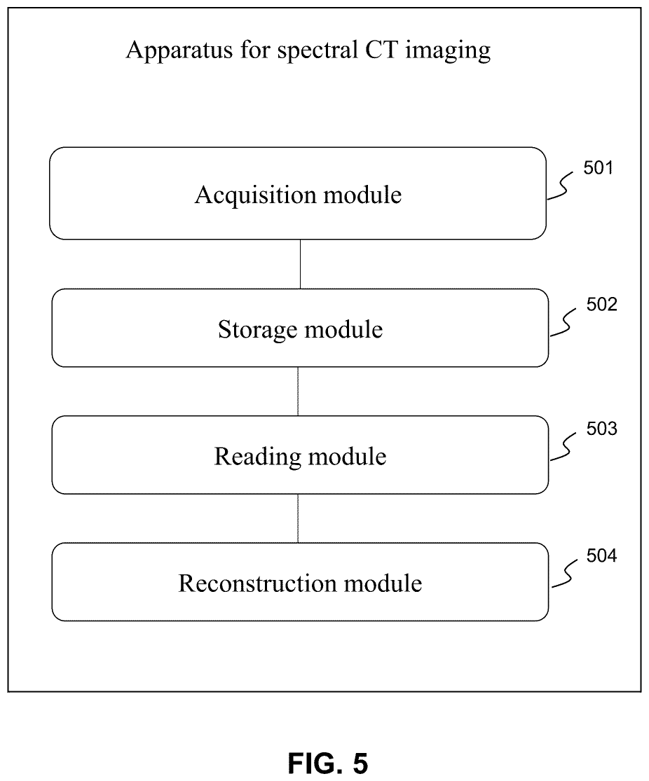

In an embodiment, referring to FIG. 5, an apparatus 500 for spectral CT imaging is provided, may include: an acquisition module 501, configured to obtain a plurality of data frames collected by a detector; a storage module 502, configured to cache each of the data frames into memory in an energy-segmented manner, and thereafter storing the data frames in the memory to a storage device in an energy-segmented manner; a reading module 503 configured to read, from the memory or the storage device, data frames required for image reconstruction; and a reconstruction module 504 configured to generate a reconstructed image by using the data frames that have been read.

In an embodiment, the reading module 503 is further configured to: when the data frames required for image reconstruction reside in the memory, read the data frames required for image reconstruction from the memory; and when the data frames required for image reconstruction do not reside in the memory, read the data frames required for image reconstruction from the storage device.

In an embodiment, the reconstruction module 504 is further configured to: store the read data frames into shared memory; block-read the data frames residing in the shared memory to obtain data frames at a plurality of angular positions; and perform reconstruction processing on the data frames at the plurality of angular positions to obtain the reconstructed image.

In an embodiment, the reconstruction module 504 is further configured to: in a first pipeline process, perform reconstruction processing on first-energy-segment data frames at the plurality of angular positions to obtain a first-energy-segment reconstructed image; and in a second pipeline process, perform reconstruction processing on second-energy-segment data frames at the plurality of angular positions to obtain a second-energy-segment reconstructed image; wherein the first pipeline process and the second pipeline process are independent of each other.

In an embodiment, the reconstruction module 504 is further configured to: in the first pipeline process, perform correction processing on the first-energy-segment data frames at the plurality of angular positions; and after the correction processing, in the first pipeline process perform reconstruction processing on the first-energy-segment data frames at the plurality of angular positions to obtain the first-energy-segment reconstructed image.

In an embodiment, the apparatus further includes a fusion module configured to: in the first pipeline process, based on fusion processing of a plurality of first-energy-segment data frames at a same angular position, obtain fused first-energy-segment data frames at each angular position.

In an embodiment, fusion processing for different angular positions is performed in parallel.

In an embodiment, the apparatus further includes a projection-image processing module configured to: based on the data frames at a same angular position read from the memory or the storage device, sequentially display projection images corresponding to the respective angular positions or a subset of the angular positions.

In an embodiment, the projection-image processing module is further configured to: based on first-angular-position data frames read from the memory or the storage device, perform correction processing on the first-angular-position data frames, to obtain and display a projection image corresponding to the first angular position; and based on second-angular-position data frames read from the memory or the storage device, perform correction processing on the second-angular-position data frames, to obtain and display a projection image corresponding to the second angular position.

Specific limitations of the spectral CT imaging apparatus may refer to the limitations of the spectral CT imaging method described above, and may not be repeated here. The various modules of the above spectral CT imaging apparatus may be implemented in whole or in part by software, hardware, or a combination thereof. The above modules may be embedded in a processor in hardware form, or may be stored in a memory of a computer device in software form, so as to be invoked by the processor to perform operations corresponding to the modules.

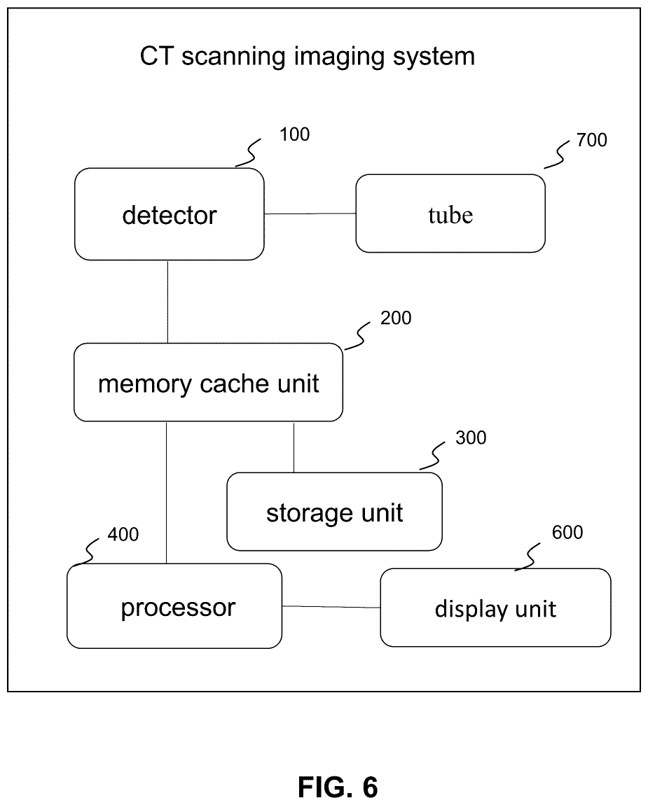

Referring to FIG. 6, In an embodiment, a CT scanning imaging system is provided, may include a detector 100, a memory cache unit 200, a storage unit 300 and a processor 400. In some embodiments, the CT scanning imaging system further includes an X-ray tube 700 for generating X-rays. In some embodiments, the CT scanning imaging system may be a CT device alone, or may be a multi-modality scanning imaging system in which a CT device is combined with other imaging devices, for example, a PET device (PET stands for positron emission tomography). In some embodiments, the CT scanning imaging system is a micro-CT scanning imaging system, which may be used for pre-clinical research analysis.

Wherein, the detector 100 may be a photon-counting detector capable of sensing X-rays, and is configured to collect data frames after scanning.

The memory cache unit 200 is configured to cache the data frames in an energy-segmented manner.

The storage unit 300 is configured to store, in an energy-segmented manner, the data frames cached in the memory cache unit.

The processor 400 is configured to perform image reconstruction using data frames read from the memory cache unit or the storage unit.

Wherein, the memory cache unit may be a physical memory storage, such as memory. Memory is an important component in a computer and is a bridge for communication with the processor. Computer programs run in memory, so the performance of memory has a great impact on the computer. Memory is also referred to as internal memory, and its role is to temporarily store operation data in the CPU and data exchanged with external storage devices such as hard disks. When a computer is running, the CPU transfers data to be operated into memory for operation, and after the operation is completed, the CPU transfers the result out. The operation of memory also determines the stable operation of the computer.

From a hardware perspective, a part of the above memory cache unit may be shared memory. Shared memory and the memory cache unit are of the same type of storage device and can be understood as physical memory, but their roles are different. Shared memory allows two unrelated processes to access the same logical memory and is a very efficient way for two running processes to share and transfer data. Memory shared between different processes is usually the same physical memory. Processes may attach the same physical memory to their own address spaces, and all processes may access the addresses in the shared memory. If one process writes data to the shared memory, the changes may immediately affect any other process that may access the same shared memory.

The storage unit may be a disk. A disk is one of the most important storage devices in a computer. Most software required for normal operation of a computer is stored on a disk. Disk storage has a larger capacity, which is different from memory. A disk is a storage device on a computer that is based on a hard rotating disk. It stores and retrieves digital data on a flat magnetic surface. In some embodiments, the disk storage unit includes a hard disk, which is a type of disk, such as a mechanical hard disk. In addition, the storage unit may also include, for example, a solid-state drive.

Further, when data frames required for image reconstruction reside in the memory cache unit, the processor reads the data frames required for image reconstruction from the memory cache unit. When data frames required for image reconstruction do not reside in the memory cache unit, the processor reads the data frames required for image reconstruction from the storage unit.

Further, when the processor generates a reconstructed image using the read data frames, the following steps are specifically performed: the processor stores the read data frames into shared memory; block-reads the data frames residing in the shared memory to obtain data frames at a plurality of angular positions; and performs reconstruction processing on the data frames at the plurality of angular positions to obtain the reconstructed image.

Further, when the processor performs reconstruction processing on the data frames at the plurality of angular positions to obtain the reconstructed image, the following steps are specifically performed: in a first pipeline process, performs reconstruction processing on first-energy-segment data frames at the plurality of angular positions to obtain a first-energy-segment reconstructed image; in a second pipeline process, performs reconstruction processing on second-energy-segment data frames at the plurality of angular positions to obtain a second-energy-segment reconstructed image; wherein the first pipeline process and the second pipeline process are independent of each other.

Further, when the processor in the first pipeline process performs reconstruction processing on the first-energy-segment data frames at the plurality of angular positions to obtain the first-energy-segment reconstructed image, the following steps are specifically performed: in the first pipeline process, performs correction processing on the first-energy-segment data frames at the plurality of angular positions; after the correction processing, in the first pipeline process performs reconstruction processing on the first-energy-segment data frames at the plurality of angular positions to obtain the first-energy-segment reconstructed image.

Further, before the processor in the first pipeline process performs correction processing on the first-energy-segment data frames at the plurality of angular positions, the following steps are further performed: in the first pipeline process, based on fusion processing of a plurality of first-energy-segment data frames at a same angular position, obtains fused first-energy-segment data frames at each angular position.

Further, fusion processing for different angular positions is performed in parallel.

Further, the CT scanning imaging system further includes a display unit 600 configured to display the reconstructed image or projection images, the display unit 600 specifically may be a monitor.

The processor may sequentially display, on the display unit, projection images corresponding to the respective angular positions or a subset of the angular positions, based on the data frames at the same angular position read from the memory cache unit or the storage unit.

Further, the processor may perform the following steps: based on first-angular-position data frames read from the memory cache unit or the storage unit, perform correction processing on the first-angular-position data frames, to obtain a projection image corresponding to the first angular position, and display the projection image corresponding to the first angular position on the display unit; based on second-angular-position data frames read from the memory cache unit or the storage unit, perform correction processing on the second-angular-position data frames, to obtain a projection image corresponding to the second angular position, and display the projection image corresponding to the second angular position on the display unit.

In an embodiment, a computer-readable storage medium is provided, storing a computer program. The computer program, when executed by a processor, causing the processor to perform the spectral CT imaging method according to any one of the above embodiments.

In an embodiment, a computer program product is provided, storing a computer program. The computer program, when executed by a processor, causes the processor to perform the spectral CT imaging method according to any one of the above embodiments.

In an embodiment, a computer device is provided, may include a processor and a storage device, wherein the storage device stores a computer program. The computer program, when executed by the processor, causes the processor to perform the spectral CT imaging method according to any one of the above embodiments.

Those of ordinary skill in the art may appreciate that all or part of the steps in the methods of the above embodiments may be implemented by instructing relevant hardware through a computer program. The computer program may be stored in a non-volatile computer-readable storage medium, and when executed, the computer program may include the steps of the method embodiments described above.

The technical features of the above embodiments may be arbitrarily combined. For the sake of brevity, not all possible combinations of the technical features in the above embodiments are described. However, as long as there is no contradiction between the combinations of these technical features, they should be regarded as within the scope described in this specification.

The above embodiments merely represent several implementations of the present disclosure, and their descriptions are specific and detailed, but should not therefore be construed as limiting the scope of the patent. It should be noted that for those of ordinary skill in the art, without departing from the spirit of the present disclosure, various modifications and improvements may be made, which all fall within the protection scope of the present disclosure. Therefore, the protection scope of the present disclosure patent should be subject to the appended claims.

What is claimed is:

1. A method for spectral CT imaging, includes:
obtaining a plurality of data frames collected by a detector;
caching each of the data frames into memory in an energy-segmented manner, and thereafter storing the data frames in the memory to a storage device in an energy-segmented manner;
reading, from the memory or the storage device, data frames required for image reconstruction; and
generating a reconstructed image using the data frames that have been read.

2. The method of claim 1, wherein reading, from the memory or the storage device, data frames required for image reconstruction includes:
when the data frames required for image reconstruction reside in the memory, reading the data frames required for image reconstruction from the memory; and
when the data frames required for image reconstruction do not reside in the memory, reading the data frames required for image reconstruction from the storage device.

3. The method of claim 1 or 2, wherein generating a reconstructed image by using the data frames that have been read includes:
storing the read data frames into shared memory;
performing block-wise reading of the data frames in the shared memory to read at least one data frame at a time, thereby obtaining data frames for a plurality of projection angles; and
reconstructing the data frames for the plurality of projection angles to obtain the reconstructed image.

4. The method of claim 3, wherein the data frames for the plurality of projection angles include first-energy-segment data frames for the plurality of projection angles and second-energy-segment data frames for the plurality of projection angles, and wherein reconstructing the data frames for the plurality of projection angles to obtain the reconstructed image includes:
reconstructing, by a first pipeline process, the first-energy-segment data frames for the plurality of projection angles to obtain a first-energy-segment reconstructed image; and
reconstructing, by a second pipeline process, the second-energy-segment data frames for the plurality of projection angles to obtain a second-energy-segment reconstructed image,
wherein the first pipeline process and the second pipeline process operate independently of each other.

5. The method of claim 4, wherein reconstructing, by the first pipeline process, the first-energy-segment data frames for the plurality of projection angles to obtain the first-energy-segment reconstructed image includes:
performing, by the first pipeline process, a correction process on the first-energy-segment data frames for the plurality of projection angles; and
after the correction process, reconstructing, by the first pipeline process, the first-energy-segment data frames for the plurality of projection angles to obtain the first-energy-segment reconstructed image.

6. The method of claim 5, before the step wherein performing, by the first pipeline process, the correction process on the first-energy-segment data frames for the plurality of projection angles, further includes:
performing, by the first pipeline process, a fusion process on a plurality of first-energy-segment data frames of a same projection angle to obtain, for each projection angle, a fused first-energy-segment data frame.

7. The method of any one of claims 4-6, wherein the first pipeline process includes a plurality of threads configured to perform, in parallel, the fusion process on first-energy-segment data frames corresponding to different projection angles.

8. The method of claim 3, wherein the data frames for the plurality of projection angles include data frames corresponding to a plurality of energy segments for the plurality of projection angles, and wherein reconstructing the data frames for the plurality of projection angles to obtain the reconstructed image includes:
performing, by a plurality of pipeline processes, image reconstruction on the data frames corresponding to the plurality of energy segments, thereby obtaining reconstructed images for the respective energy segments.

9. The method of any one of claims 1-8, further includes:
displaying projection images corresponding to each angle or partial angle, sequentially, based on data frames read from memory or storage device at the same angle.

10. The method of claim 9, wherein displaying projection images corresponding to each angle or partial angle, sequentially, includes:
performing a correction process on first-projection-angle data frames read from the memory or the storage device to obtain and display a projection image corresponding to the first projection angle; and
performing a correction process on second-projection-angle data frames read from the memory or the storage device to obtain and display a projection image corresponding to the second projection angle.

11. The method of any one of claims 1-10, wherein caching each of the data frames into memory in an energy-segmented manner and thereafter storing the data frames in the memory to a storage device in an energy-segmented manner includes:
while writing a first batch of the plurality of data frames into the memory, writing a second batch of the plurality of data frames from the memory to the storage device.

12. The method of any one of claims 1-11, wherein the plurality of data frames include full-energy-segment data frames and high-energy-segment data frames, the method further includes:
obtaining low-energy-segment data frames based on a difference between the full-energy-segment data frames and the high-energy-segment data frames.

13. An apparatus for spectral CT imaging, includes:
an acquisition module configured to obtain a plurality of data frames collected by a detector;
a storage module configured to cache each of the data frames into memory in an energy-segmented manner, and thereafter storing the data frames in the memory to a storage device in an energy-segmented manner;
a reading module configured to read, from the memory or the storage device, data frames required for image reconstruction; and
a reconstruction module configured to generate a reconstructed image by using the data frames that have been read.

14. A CT scanning imaging system, includes:
a detector configured to collect data frames after scanning;
a memory cache unit configured to cache the data frames in an energy-segmented manner;
a storage unit configured to store, in an energy-segmented manner, the data frames cached in the memory cache unit; and
a processor configured to perform image reconstruction using data frames read from the memory cache unit or the storage unit.

15. A computing device includes a processor and a memory storing computer program code, wherein the computer program code, when executed by the processor, causes the processor to perform the method of any one of claims 1-12.

16. A non-transitory computer-readable storage medium storing computer program code, wherein the computer program code, when executed by a processor, causes the processor to perform the method of any one of claims 1-12.

17. A computer program product storing computer program code, wherein the computer program code, when executed by a processor, causes the processor to perform the method of any one of claims 1-12.
